SEQUENCE LISTING (3 sequences)

SEQ ID NO: 1 - DNA, 2836 bp, Homo sapiens
  CDS (1)...(2136)
atggcggagcgagagagcggcggcctgggagggggggccgcgtccccgcccgccgcctccccgttcctggggctgcacatcgcttcgcctcccaatttcaggctcacccatgacatcagcctggaggagtttgaggatgaagacctctcggagatcactgatgagtgtggcatcagcttacagtgcaaagacaccctgtccttacggcccccgcgcgccgggctgctctctgcgggcggcggcggcgcggggagccggttgcaggccgagatgctgcagatggacctgatcgacgcgacgggggacactcccggggccgaggacgacgaggaggacgacgacgaggagcgcgcggcccggcggccgggagcggggccgcccaaggccgaatccggccaggagccggcgtcccgcggccagggccagagccaaggccagagccagggcccgggcagcggggacacgtaccggcccaagcggcccaccacgctcaacctctttccgcaggtgccgcggtctcaggacacactgaataataattctctgggcaaaaagcacagttggcaggatcgggtgtctcgatcatcctcacccctgaagacaggggagcagacaccaccgcatgaacacatctgcctgagcgaggagctgcccccccagagcggccccgcccccaccacagatcgaggcacctccaccgacagcccttgccgccgcagcacagccacccagatggcacctccgggtggtccccctgctgccccgcctgggggtcggggccactcgcatcgagaccgaatccactaccaggccgatgtgcgactagaggccactgaggagatctacctgaccccagtgcagaggcccccagacgctgcagagcccacctccgccttcctgccgcccactgagagccggatgtcagtcagctccgatccagaccctgccgcctacccctccacggcagggcggccgcacccctccatcagtgaagaggaagagggcttcgactgcctgtcgtccccagagcgggctgagcccccaggcggagggtggcgggggagcctgggggagccgccgccacctccacgggcctctctgagctcggacaccagcgccctgtcctatgactctgtcaagtacacgctggtggtagatgagcatgcacagctggagctggtgagcctgcggccgtgcttcggagactacagtgacgagagtgactctgccaccgtctatgacaactgtgcctccgtctcctcgccctatgagtcggccatcggagaggaatatgaggaggccccgcggccccagccccctgcctgcctctccgaggactccacgcctgatgaacccgacgtccatttctccaagaaattcctgaacgtcttcatgagtggccgctcccgctcctccagtgctgagtccttcgggctgttctcctgcatcatcaacggggaggagcaggagcagacccaccgggccatattcaggtttgtgcctcgacacgaagacgaacttgagctggaagtggatgaccctctgctagtggagctccaggctgaagactactggtacgaggcctacaacatgcgcactggtgcccggggtgtctttcctgcctattacgccatcgaggtcaccaaggagcccgagcacatggcagccctggccaaaaacagtgactgggtggaccagttccgggtgaagttcctgggctcagtccaggttccctatcacaagggcaatgacgtcctctgtgctgctatgcaaaagattgccaccacccgccggctcaccgtgcactttaacccgccctccagctgtgtcctggagatcagcgtgcggggtgtgaagataggcgtcaaggccgatgactcccaggaggccaaggggaataaatgtagccactttttccagttaaaaaacatctctttctgcggatatcatccaaagaacaacaagtactttgggttcatcaccaagcaccccgccgaccaccggtttgcctgccacgtctttgtgtctgaagactccaccaaagccctggcagagtccgtggggagagcattccagcagttctacaagcagtttgtggagtacacctgccccacagaagatatctacctggagtagctgtgcagccccgccctctgcgtcccccagccctcaggccagtgccaggacagctggctgctgacaggatgtggcactgcttgaggaggggcacctgccaccgccagaggacaaggaagtgggggccgctggcccagggtaggggagggtggggcaatggggagaggcaaatgcagtttattgtaatatatgggattagattcatctatggagggcagagtgggctgcctggggattgggagggacagggcttggggagcaggtctctggcagagaaggatgtccgttccaggagcacacggccctgccccatcctgggccttacctcccctgccagggctcgggcgctgtggctcctgccttgatgaagcccgtgtcctgccttgatgaagcctgtgccacctgcaagtgcccgccctgcccctgcccaacccccgaagaccctgagctcaggctgagcccagccacctcccaaggactttccagtaggaaatggcaacaggtgaagtccctgttctcagctccgtcatctggggtcgggggctcctgccactgacctcaccggcatgctggcctgtggcaggcctaggacctcaggcggggaggaggagcgccagcccgtccgaaaaggaggcttctgcgaccggcaccctctggcctgccctggccaatttaaagtgcatttcctgtcaaaaaaaaa

SEQ ID NO: 2 - protein, 711 aa, Homo sapiens
MAERESGGLGGGAASPPAASPFLGLHIASPPNFRLTHDISLEEFEDEDLSEITDECGISLQCKDTLSLRPPRAGLLSAGGGGAGSRLQAEMLQMDLIDATGDTPGAEDDEEDDDEERAARRPGAGPPKAESGQEPASRGQGQSQGQSQGPGSGDTYRPKRPTTLNLFPQVPRSQDTLNNNSLGKKHSWQDRVSRSSSPLKTGEQTPPHEHICLSEELPPQSGPAPTTDRGTSTDSPCRRSTATQMAPPGGPPAAPPGGRGHSHRDRIHYQADVRLEATEEIYLTPVQRPPDAAEPTSAFLPPTESRMSVSSDPDPAAYPSTAGRPHPSISEEEEGFDCLSSPERAEPPGGGWRGSLGEPPPPPRASLSSDTSALSYDSVKYTLVVDEHAQLELVSLRPCFGDYSDESDSATVYDNCASVSSPYESAIGEEYEEAPRPQPPACLSEDSTPDEPDVHFSKKFLNVFMSGRSRSSSAESFGLFSCIINGEEQEQTHRAIFRFVPRHEDELELEVDDPLLVELQAEDYWYEAYNMRTGARGVFPAYYAIEVTKEPEHMAALAKNSDWVDQFRVKFLGSVQVPYHKGNDVLCAAMQKIATTRRLTVHFNPPSSCVLEISVRGVKIGVKADDSQEAKGNKCSHFFQLKNISFCGYHPKNNKYFGFITKHPADHRFACHVFVSEDSTKALAESVGRAFQQFYKQFVEYTCPTEDIYLE

SEQ ID NO: 3 - protein, 19 aa, Homo sapiens
GVKIGVKADDSQEAKGNKC

(19) United States
(12) Patent Application Publication
(10) Pub. No.: US 2002/0090696 A1
(43) Pub. Date: Jul. 11, 2002

(54) Treating neurological disorders using human apoptosis inhibiting protein

(76) Inventors: Carol A. Miller, San Marino, CA (US); Zhao Hui Dong, Torrence, CA (US); Yan Zhang-Klompus, Redwood City, CA (US)

Correspondence Address: Rajiv Yadav, McCutchen, Doyle, Brown & Enersen, LLP, Three Embarcadero Center, 18th Floor, San Francisco, CA, 94111, US

(21) Appl. No.: 09/966,561

(22) Filed: Sep. 27, 2001

Related U.S. Application Data
(63) Continuation of application No. 09/419,694, filed on Oct. 14, 1999.
(60) Provisional application No. 60/111,502, filed on Dec. 8, 1998.

(51) Int. Cl. A61K 38/17; C12N 9/99

(52) U.S. Cl. 435/184; 514/12

(57) ABSTRACT

The invention relates to methods of using full length amino acid sequence and cDNA sequence of protein human JNK interacting protein (hJ1P1) islet brain 1 (IB1) to treat neurological disorders in human subjects. In an in vitro model of the apoptosis pathway, HJIP-1/IB1 inhibits phosphorylation of the transcription factor c-Jun in a dose-dependent manner. The invention includes antibodies that specifically bind to hJIP-1/IB1.

### RELATED APPLICATION DATA

This application is a continuation of U.S. patent application Ser. No. 09/419,694, filed Oct. 14, 1999, which claims priority to provisional application Ser. No. 60/111,502, filed Dec. 8, 1998.

### GOVERNMENT SUPPORT

The U.S. Government has certain rights in this invention pursuant to National Institutes of Health grant numbers 5R37MH39145, 5P50AG05142 and 5K12AG00521.

### FIELD OF THE INVENTION

The present invention provides novel polynucleotides and proteins encoded by such polynucleotides. This invention relates generally to the field of protein kinases, and specifically to a protein that binds to and inhibits phosphorylation of c-Jun by the c-Jun amino-terminal kinase (JNK).

### BACKGROUND

Oxidative stress-induced damage is implicated in several human diseases, particularly in stroke and more chronically in neurodegenerative diseases, such as Alzheimer's disease, Parkinson's disease and amyotrophic lateral sclerosis (Lou Gehrig's disease). JNKs, especially JNK3, a neuron-specific, stress-activated protein kinase may be central respondents to oxidant stresses, including Aβ, hypoxia or ischemia as well as glutamate excitotoxicity (FIG. 1). JNK is one member of the stress-activated protein kinase (SAPK) signal transduction pathway that specifically respond to a variety of environmental and cellular stress factors. Once activated, JNK3 may trigger cell death mechanisms of apoptosis by phosphorylation of the transcription factor c-Jun and by interactions with other cellular proteins, such as tau (Reynolds et al., 1997) and DENN-MADD, a GDP-GTP exchange protein (Zhang et al., 1998) further contributing to cellular dysfunction. These changes may occur rapidly after stress induction, potentially during an early and yet reversible injury stage. JNK3 phosphorylates tau protein. This phosphorylation may be one step towards formation of abnormal tau protein found in Alzheimer's disease and several other dementing diseases.

Proteins have been found to regulate JNK in mice and rats. These proteins have been called c-Jun interacting protein (JIP) or islet-brain 1 (IB1). Murine protein, JIP-1 was reported as a murine JNK1 interacting protein (2). JIP-1 causes cytoplasmic retention of JNK1 and inhibition of JNK-regulated gene expression in non-neural (COS) cells. Moreover, preliminary observations suggested that JIP-1 can greatly reduce the nerve growth factor (NGF) withdrawal-induced cell death in PC12 rat peripheral neuron cultures. Bonny et al. (1998) identified IB1 as a 714 amino-acid transcriptional activator from a rat cDNA expression library. Rat homologs of JIP1/IB1 interact with all three JNK classes (Bonny et al., 1997; Dickens et al., 1997; Elion, 1998).

U.S. Pat. No. 6,043,083 (the “'083 Patent”) describes the identification of murine JIP-1 proteins that interact with JNK but does not disclose the identification of human JIP proteins speculating instead that human homologs of the murine JIP-1 gene can be isolated using molecular biology methods. Further, the '083 Patent focuses on JIP-1's inhibition of JNK-regulated signal transduction in transfected CHO cells and inhibition of pre-B cell transformation. Additionally, although the '083 Patent provides data to show that overexpression of JIP-1 inhibits NGF withdrawal-induced apoptosis of PC-12 cells, it does not provide any data of direct therapeutic intervention of neurodegenerative diseases such as Alzheimer's disease using JNK inhibiting proteins.

Published PCT application no. 98/00972 (the “'972 Application”) discloses a human IB1 protein, which is similar to the hJIP-1/IB1 protein disclosed herein. However, the '972 application focuses on IB1's transcriptional ability in regulating GLUT2 expression in pancreatic beta-cells and primarily suggests the use of IB1 to treat diabetes. The '972 Application speculates that IB1 may be useful for treating neurodegenerative disease but provides no data whatsoever to support that hypothesis.

There exists a need to establish the usefulness of JNK inhibiting proteins in neurodegenerative disease, especially in the human model.

### SUMMARY OF THE INVENTION

The invention describes the polynucleotide sequence of the cDNA of human JNK interacting protein hJIP-1/IB1. The invention also describes the deduced amino acid sequence of the protein hJIP-1/IB1. The invention also describes isolated hJIP-1/IB1 proteins.

hJIP-1/IB1 interacts with and is phosphorylated by JNK. hJIP-1/IB1 inhibits the activation of c-Jun by JNK3, the neuron-specific JNK. The invention includes methods for inhibiting JNK3.

The invention describes antibodies that specifically interact with hJIP-1/IB1 . In one embodiment, a polyclonal antisera specific for residues 617-635 of protein hJIP-1/IB1 (SEQ ID NO: 3) is described.

Polynucleotides include those with sequences substantially equivalent to SEQ ID NO: 1, including fragments thereof. Polynucleotides of the present invention also include, but are not limited to, a polynucleotide complementary to the nucleotide sequence of SEQ ID NO: 1.

Polynucleotides according to the invention have numerous applications in a variety of techniques known to those skilled in the art of molecular biology. These techniques include use as hybridization probes, use as oligomers for PCR, use for chromosome and gene mapping, use in the recombinant production of protein, and use in generation of antisense DNA or RNA, their chemical analogs and the like. For example, when the expression of an mRNA is largely restricted to a particular cell or tissue type, polynucleotides of the invention can be used as hybridization probes to detect the presence of the particular cell or tissue mRNA using, e.g., in situ hybridization.

The polypeptides according to the invention can be used in a variety of procedures and methods that are currently applied to other proteins. For example, a polypeptide of the invention can be used to generate an antibody that specifically binds the polypeptide. The polypeptides of the invention also serve as substrates for JNKs and can therefore be used for the study of JNKs. The polypeptides of the invention also inhibit the ability of JNKs to phosphorylate c-Jun and can, therefore, be used to study the phosphorylation of c-Jun by JNKs.

Methods are also provided for preventing, treating or ameliorating a medical condition which comprises administering to a mammalian subject a therapeutically effective amount of a composition comprising a protein of the present invention or a polynucleotide of the present invention and a pharmaceutically acceptable carrier. In particular, the polypeptides and polynucleotides of the invention can be utilized, for example, as part of methods for modulation of the oxidative stress response in neuronal tissue. The polypeptides and polynucleotides of the invention may, therefore, be utilized, for example, as part of methods for treatment of Alzheimer's disease, Parkinson's disease and amyotrophic lateral sclerosis (Lou Gehrig's disease). In one embodiment, a polynucleotide of the invention is delivered to a patient in an appropriate vector that directs expression of the polypeptide in neuronal tissue.

In specific embodiments, the invention provides a method of treating a neurological disorder in a human patient which comprises administering to said human patient an effective amount of a composition comprising a polypeptide comprising a sequence substantially equivalent to SEQ ID NO: 2. The composition may further comprise a pharmaceutically acceptable carrier. Preferably, the composition is administered orally, transdermally, intravenously, intrasynovially, intramuscularly, intraocularly, intranasally, intrathecally, or topically. In alternative embodiment, the composition of the invention is administered in conjunction with another method of treating a neurological disorder.

The neurological disorder may be caused by oxidative stress response in neuronal tissue. It may be caused by the activation of a neuron specific, stress-activated protein kinase, such as c-Jun amino-terminal kinase 3. Specifically, the neurological disorder is a disorder selected from dementia, dementia of the Alzheimer's type, bipolar disorders, mood disorder with depressive features, mood disorder with major depressive-like episode, mood disorder with manic features, mood disorder with mixed features, substance-induced mood disorder and mood disorder not otherwise specified (NOS), panic disorder without agoraphobia, panic disorder with agoraphobia, agorathobia without history of panic disorder, social phobia, postraumatic stress disorder, acute stress disorder, substance-induced anxiety disorder and anxiety disorder not otherwise specified (NOS), dyskinesias and behavioral manifestations of mental retardation, conduct disorder and autistic disorder. The dementia may be dementia selected from the group consisting of vascular dementia, dementia due to HIV disease, dementia due to head trauma, dementia due to Parkinson's disease, dementia due to Huntington's disease, dementia due to Pick's disease, dementia due to Creutzfeldt-Jakob disease, substance-induced persisting dementia, dementia due to multiple etiologies and dementia not otherwise specified (NOS).

Alternatively, the dementia is dementia of the Alzheimer's type, which may be selected from the group consisting of dementia of the Alzheimer's type with early onset uncomplicated, dementia of the Alzheimer's type with early onset with delusions, dementia of the Alzheimer's type with early onset with depressed mood, dementia of the Alzheimer's type with late onset uncomplicated, dementia of the Alzheimer's type with late onset with delusions and dementia of the Alzheimer's type with late onset with depressed mood.

Preferably, the composition is administered in a targeted drug delivery system, for example, a targeted drug delivery system such as a liposome coated with an antibody that specifically targets neuronal tissue.

The invention also provides a method of treating a neurological disease in a human subject selected from the group consisting of Alzheimer's disease, stroke, amyotrophic lateral sclerosis, age associated memory impairment and Parkinson's disease, the method comprising administering to said human an effective amount of a composition comprising a polypeptide having a sequence that is substantially equivalent to SEQ ID NO: 2. The composition may further comprise a pharmaceutically acceptable carrier. Preferably, the composition is administered orally, transdermally, intravenously, intrasynovially, intramuscularly, intraocularly, intranasally, intrathecally, or topically. In alternative embodiment, the composition of the invention is administered in conjunction with another method of treating a neurological disorder.

The invention also provides for a method of treating Alzheimer's disease, stroke, amyotrophic lateral sclerosis, age associated memory impairment or Parkinson's disease in a human subject, the method comprising administering to said human an effective amount of a composition comprising a polynucleotide having a sequence that is substantially equivalent to SEQ ID NO: 1. Preferably, this composition is administered to the subject's cells using a recmobinant expression vector that comprises a sequence substantially equivalent to SEQ ID NO: 1. Administering the composition may further comprise removing stem cells from a subject's bone marrow; introducing the recombinant expression vector into the removed stem cells; and re-introducing the stem cells into the subject's bone marrow.

The invention also provides a method of treating a neurological disease in a human subject selected from the group consisting of Alzheimer's disease, stroke, amyotrophic lateral sclerosis, age associated memory impairment and Parkinson's disease, the method comprising administering to said human an effective amount of a composition comprising a polypeptide having a sequence that is substantially equivalent to SEQ ID NO: 2. The composition may further comprise a pharmaceutically acceptable carrier. Preferably, the composition is administered orally, transdermally, intravenously, intrasynovially, intramuscularly, intraocularly, intranasally, intrathecally, or topically. In alternative embodiment, the composition of the invention is administered in conjunction with another method of treating a neurological disorder.

The methods of the present invention further relate to the methods for detecting the presence of the polynucleotides or polypeptides of the invention in a sample. Such methods can, for example, be utilized as a prognostic indicator of stroke or Alzheimer's disease, Parkinson's disease and amyotrophic lateral sclerosis (Lou Gehrig's disease).

### BRIEF DESCRIPTION OF THE DRAWINGS

FIG. 1 illustrates a schematic model of stress induction of JNK/SAPKs and shows the convergence and transduction of external factors, including oxidant, excitotoxic and inflammatory agents on the JNK/SAPK system. Downstream targets phosphorylated by JNK transduction of stress stimuli include tau, a class of neuronal cytoskeletal proteins that form the core of neurofibrillary tangles in Alzheimer's disease, DENN/MADD which may affect nucleolar function, and transcription factors, especially c-Jun which may translocate into the nucleus and participate in apoptosis.

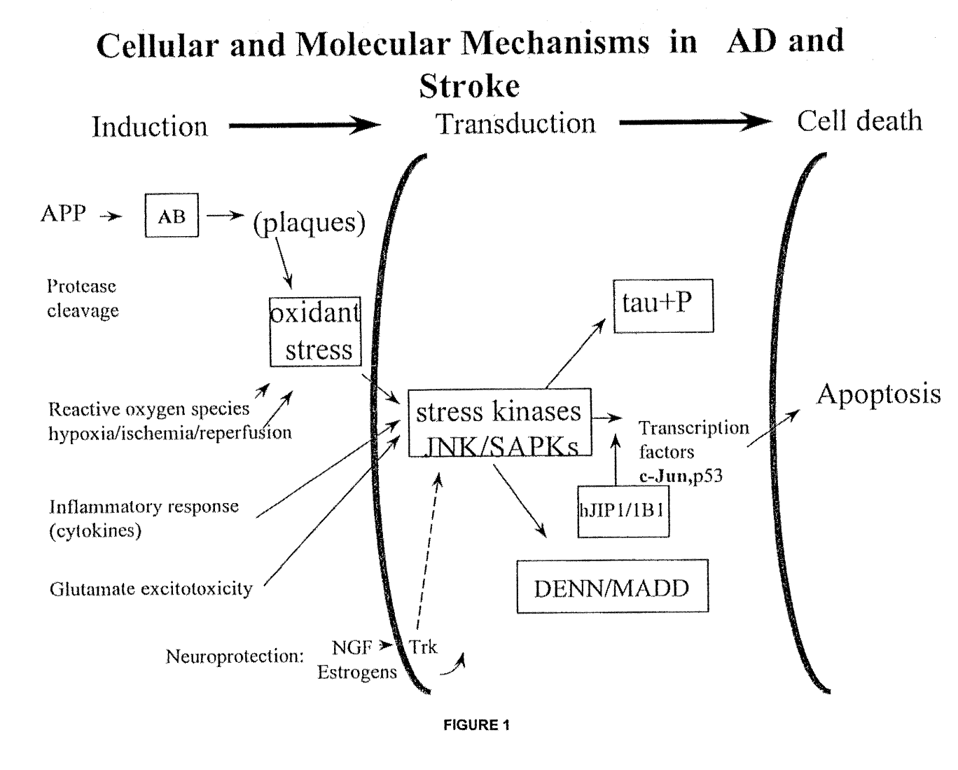

FIG. 2 shows a domain map of hJIP1/IB1 (SEQ ID NO: 2) and clone 2.5 (corresponding to amino acid residues 76-711 of SEQ ID NO: 2). The JNK binding domain is found in the region from about residue 127 to 285.

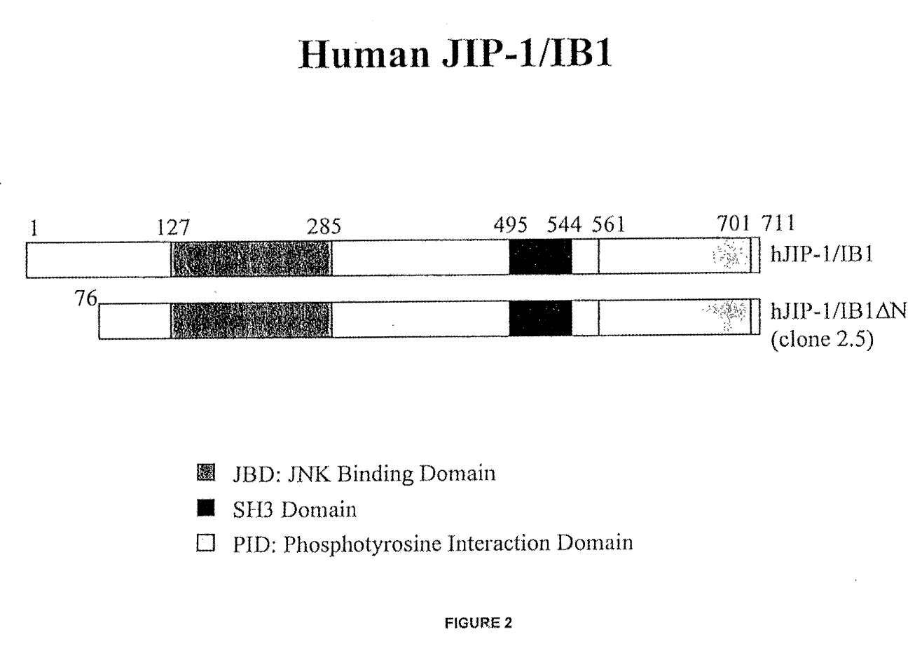

FIG. 3 shows that truncated hJIP-1/IB1 (protein encoded by clone 2.5 or “protein 2.5,” corresponding to residues 76-711 of SEQ ID NO: 2) interacts with JNK3 in vitro. A. Clone 2.5 cDNA (corresponding to nucleotides 226-2836 of SEQ ID NO: 1) was subcloned into EcoRI and XhoI sites of pGEX-4T-2 vector (Pharmacia Biotech, Inc.) in-frame with GST moiety. GST-2.5 fusion protein was expressed and purified from E. coli host DH5α. An in vitro kinase assay was performed using GST-c-Jun as control. Rat SAPKβ (GenBank Accession No. L27128 (mRNA sequence); GenBank Accession No. AAA42110 (protein sequence)), which has virtually complete identity with JNK3, phosphorylates protein 2.5. B. clone 2.5 inhibits c-Jun phosphorylation: In a kinase reaction with c-Jun and rSAPKβ, protein 2.5 shows a dose-dependent inhibition of phosphorylation of GST-c-Jun. GST alone shows no effect. Inhibition of JNK3 phosphorylation of c-Jun by protein 2.5 suggests that the latter is a potential inhibitor of the JNK pathway in vivo. Theoretically, binding of protein 2.5 to JNK3 will block the activation sites for c-Jun phosphorylation, or make those sites less accessible by changing the conformation of JNK3.

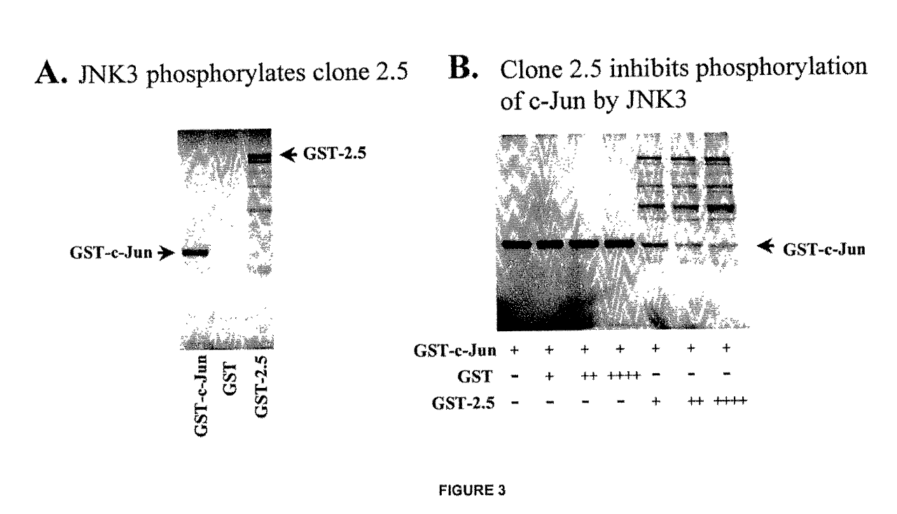

FIG. 4 shows multiple tissue Northern analysis using clone 2.5 as a probe (nucleotides 226-2836 of SEQ ID NO: 1). Analysis reveals strong signals of 3.0 and 3.3 kb in brain and 3.0 kb in pancreas. In lower panel, actin cDNA is used as a loading control.

FIG. 5 illustrates the tissue distribution of hJIP1/IB1. Control and hypoxic brain tissue (immunocytochemistry): A. Normal rat hippocampus inununostained with anti-hJIP1/IB1 polyclonal antisera shows the most intense staining in CA2 and CA3, (arrow). B. Human cerebellum: Normal cerebellum reveals cytoplasmic staining of Purkinje cell somas (arrows); (hematoxylin counterstain). C. Human cerebellum: Hypoxic changes reveal absence of anti-hJIP1/IB1 polyclonal antisera immunoreactivity of Purkinje cells. In underlying white matter, axons are immunostained (far right); (hematoxylin counterstain).

FIG. 6 shows anti hJIP1/IB1 antibody specifically recognizes hJIP1/IB1. A. Immunoblot of purified GST-2.5 fusion protein is recognized by anti-hJIP1/IB1 polyclonal antibody, but GST moiety (˜27 kD) alone is not. B. N₂A cell lysates transfected with a Xpress-tagged clone 2.5 cDNA were immunoprecipitated with anti-hJIP1/IB1 and detected with anti-Xpress. Mock transfected culture served as control. C. N₂A cells transfected with an Xpress-tagged clone 2.5 cDNA were immunostained with either anti-serum or pre-immune serum. Only anti-serum detected transfected N₂A cells.

FIG. 7 shows the protocol for hypoxic induction and activation of JNK3 and apoptosis. A model to test hJIP1/IB1 inhibition of cell death in rat organotypic hippocampal cultures is established.

FIG. 8 shows hypoxia-induced apoptosis of selectively vulnerable neurons in rat organotypic cultures. A. Low magnification reveals loss of neurons in CA1 and preservation in CA3 after 2-3 hrs. hypoxia. B. At higher magnification, neurons in CA1 show typical eosinophilic (red) cytoplasm, an early hypoxic change (hematoxylin and eosin). C. After 4 hrs. hypoxia, plus 20 hrs. re-oxygenation, there is severe edema and abundant stained “red” neurons. D. A normal control shows intact neurons of CA1 (hematoxylin and eosin).

FIG. 9 shows rat organotypic hippocampal cultures before and after hypoxia. Immunoblots include samples harvested at 0 hrs. (normal), 4 hours hypoxia, or 4 hours hypoxia followed by 20 hrs. reperfusion. In upper panel, note enhanced 45 and 52kD bands detected by anti-phosphoJNK, an indicator of JNK activation. The lower panel shows c-Jun activation detected by anti-phospho-c-Jun of hypoxia-treated cultures only.

### DETAILED DESCRIPTION

### DEFINITIONS

The term “nucleotide sequence” refers to a heteropolymer of nucleotides or the sequence of nucleotides. One of skill in the art will readily discern from contextual cues which of the two definitions is appropriate. The terms “nucleic acid” and “polynucleotide” are also used interchangeably herein to refer to a heteropolymer of nucleotides. Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “oligonucleotide fragment” or a “polynucleotide fragment,” “portion,” or “segment” refer to a stretch of nucleotide residues which is long enough to use in polymerase chain reaction (PCR) or various hybridization procedures to identify or amplify identical or related parts of mRNA or DNA molecules.

“Oligonucleotides” or “nucleic acid probes” are prepared based on the polynucleotide sequences provided herein. Oligonucleotides comprise portions of such a polynucleotide sequence having at least about 15 nucleotides and usually at least about 20 nucleotides. Nucleic acid probes comprise portions of such a polynucleotide sequence having fewer nucleotides than about 6 kb, usually fewer than 1 kb. After appropriate testing to eliminate false positives, these probes may, for example, be used to determine whether specific mRNA molecules are present in a cell or tissue.

The term “probes” includes naturally occurring or recombinant or chemically synthesized single- or double-stranded nucleic acids. They may be labeled by nick translation, Klenow fill-in reaction, PCR or other methods well known in the art. Probes of the present invention, their preparation and/or labeling are elaborated in Sambrook, J. et al., 1989. Molecular Cloning: A Laboratory Manual, Cold Spring Harbor, New York; or Ausubel, F. et al., 1989, Current Protocols in Molecular Biolog, John Wiley & Sons, New York, both of which are incorporated herein by reference in their entirety.

The term “recombinant,” when used herein to refer to a polypeptide or protein, means that a polypeptide or protein is derived from recombinant (e.g. microbial, mammalian, or insect-based) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “recombinant expression vehicle or vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product.

The term “recombinant expression system” means host cells which have stably integrated a recombinant transcriptional unit into chromosomal DNA or carry the recombinant transcriptional unit extrachromosomally. Recombinant expression systems as defined herein will express heterologous polypeptides or proteins upon induction of the regulatory elements linked to the DNA segment or synthetic gene to be expressed. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

The term “open reading frame,” or “ORF,” means a series of nucleotide triplets coding for amino acids without any termination codons and is a sequence translatable into protein.

The term “active” refers to those forms of the polypeptide which retain the biologic and/or immunologic activities of any naturally occurring polypeptide.

The term “naturally occurring polypeptide” refers to polypeptides produced by cells that have not been genetically engineered and specifically contemplates various polypeptides arising from post-translational modifications of the polypeptide including, but not limited to, acetylation, carboxylation, glycosylation, phosphorylation, lipidation and acylation.

The term “derivative” refers to polypeptides chemically modified by such techniques as ubiquitination, labeling (e.g., with radionuclides or various enzymes), pegylation (derivatization with polyethylene glycol) and insertion or substitution by chemical synthesis of amino acids such as omithine, which do not normally occur in human proteins.

The term “recombinant variant” refers to any polypeptide differing from naturally occurring polypeptides by amino acid insertions, deletions, and substitutions, created using recombinant DNA techniques. Guidance in determining which amino acid residues may be replaced, added or deleted without abolishing activities of interest, such as cellular trafficking, may be found by comparing the sequence of the particular polypeptide with that of homologous peptides and minimizing the number of amino acid sequence changes made in regions of high homology.

Preferably, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are typically in the range of about 1 to 5 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression. For example, cysteine residues can be deleted or substituted with another amino acid residue in order to eliminate disulfide bridges.

As used herein, “substantially equivalent” can refer both to nucleotide and amino acid sequences, for example a mutant sequence, that varies from a reference sequence by one or more substitutions, deletions, or additions, the net effect of which does not result in an adverse functional dissimilarity between the reference and subject sequences. Typically, such a substantially equivalent sequence varies from one of those listed herein by no more than about 2% (i.e., the number of individual residue substitutions, additions, and/or deletions in a substantially equivalent sequence, as compared to the corresponding reference sequence, divided by the total number of residues in the substantially equivalent sequence is about 0.02 or less). Such a sequence is said to have 98% sequence identity to the listed sequence. In one embodiment, a substantially equivalent, e.g., mutant, sequence of the invention varies from a listed sequence by no more than 2% (98% sequence identity); in a variation of this embodiment, by no more than 0.5% (99.5% sequence identity); and in a further variation of this embodiment, by no more than 0.1% (99.9% sequence identity). Substantially equivalent, e.g., mutant, amino acid sequences according to the invention generally have at least 98% sequence identity with a listed amino acid sequence, whereas substantially equivalent nucleotide sequence of the invention can have lower percent sequence identities, taking into account, for example, the redundancy or degeneracy of the genetic code. For the purposes of determining equivalence, truncation of the mature sequence (e.g., via a mutation which creates a spurious stop codon) should be disregarded.

Where desired, an expression vector may be designed to contain a “signal or leader sequence” which will direct the polypeptide through the membrane of a cell. Such a sequence may be naturally present on the polypeptides of the present invention or provided from heterologous protein sources by recombinant DNA techniques.

A polypeptide “fragment,” “portion,” or “segment” is a stretch of amino acid residues of at least about 5 amino acids, often at least about 7 amino acids, typically at least about 9 to 13 amino acids, and, in various embodiments, at least about 17 or more amino acids. To be active, any polypeptide must have sufficient length to display biologic and/or immunologic activity.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

The term “activated” cells as used in this application are those which are engaged in extracellular or intracellular membrane trafficking, including the export of neurosecretory or enzymatic molecules as part of a normal or disease process.

The term “purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other component normally present in a solution of the same. The terms “isolated” and “purified” do not encompass nucleic acids or polypeptides present in their natural source.

The term “open reading frame,” ORF, means a series of nucleotide triplets coding for amino acids without any termination codons and is a sequence translatable into protein.

The term “expression modulating fragment,” EMF, means a series of nucleotides which modulates the expression of an operably linked ORF or another EMF.

The term “infection” refers to the introduction of nucleic acids into a suitable host cell by use of a virus or viral vector.

The term “transformation” means introducing DNA into a suitable host cell so that the DNA is replicable, either as an extrachromosomal element, or by chromosomal integration.

The term “transfection” refers to the taking up of an expression vector by a suitable host cell, whether or not any coding sequences are in fact expressed.

Each of the above terms is meant to encompasses all that is described for each, unless the context dictates otherwise.

### Polynucleotides and Nucleic Acids of the Invention

Nucleotide and amino acid sequences of the invention are reported below. The present invention also provides genes corresponding to the cDNA sequences disclosed herein. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials.

The compositions of the present invention include isolated polynucleotides, including recombinant DNA molecules, cloned genes or degenerate variants thereof, especially naturally occurring variants such as allelic variants, novel isolated polypeptides, and antibodies that specifically recognize one or more epitopes present on such polypeptides.

### Nucleic Acids of the Invention

SEQ ID NO: 1 encodes the polypeptide sequence of SEQ ID NO: 2. In general, the high degree of amino acid residue identity between SEQ ID NO: 2 and murine or rat homologs identifies SEQ ID NO: 2 as a JNK interacting protein.

The proteins of the invention are also useful for making antibody substances that are specifically immunoreactive with hJIP-1/IB1. Antibodies and other small molecules which bind to the protein of the invention can act as blocking agents, or as activators.

In particular embodiments, the isolated polynucleotides of the invention include, but are not limited to, a polynucleotide comprising the nucleotide sequence of SEQ ID NO: 1 and nucleotides 226-2836 of SEQ ID NO: 1. The polynucleotides of the invention additionally include the complement of any of the polynucleotides recited above.

The polynucleotides of the invention also provide polynucleotides including nucleotide sequences that are substantially equivalent to the polynucleotides recited above. Polynucleotides according to the invention can have at least about 95%, more typically at least about 99%, and even more typically at least about 99.5%, sequence identity to a polynucleotide recited above. The invention also provides the complement of the polynucleotides including a nucleotide sequence that has at least about 95%, more typically at least about 99%, and even more typically at least about 99.5%, sequence identity to a polynucleotide encoding a polypeptide recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions which can routinely isolate polynucleotides of the desired sequence identities.

A polynucleotide according to the invention can be joined to any of a variety of other nucleotide sequences by well-established recombinant DNA techniques (see Sambrook J et al. (1989) Molecular Cloning: A Laboratory Manual, Cold Spring Harbor Laboratory, NY). Useful nucleotide sequences for joining to polypeptides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The sequences falling within the scope of the present invention are not limited to the specific sequences herein described, but also include allelic variations thereof. Allelic variations can be routinely determined by comparing the sequence provided in SEQ ID NO: 1, a representative fragment thereof, or a nucleotide sequence at least 98% identical to SEQ ID NO: 1 or nucleotides 226-2836 of SEQ ID NO: 1, with a sequence from another human isolate. An allelic variation is more typically at least 99% identical to SEQ ID NO: 1 and even more typically 99.8% identical to SEQ ID NO: 1. Furthermore, to accommodate codon variability, the invention includes nucleic acid molecules coding for the same amino acid sequences as do the specific ORFs disclosed herein. In other words, in the coding region of an ORF, substitution of one codon for another which encodes the same amino acid is expressly contemplated. Any specific sequence disclosed herein can be readily screened for errors by resequencing a particular fragment, such as an ORF, in both directions (i.e., sequence both strands).

The present invention further provides recombinant constructs comprising a nucleic acid having the sequence of SEQ ID NO: 1 or a fragment thereof. The recombinant constructs of the present invention comprise a vector, such as a plasmid or viral vector, into which a nucleic acid having the sequence of SEQ ID NO: 1 or a fragment thereof is inserted, in a forward or reverse orientation. In the case of a vector comprising one of the ORFs of the present invention, the vector may further comprise regulatory sequences, including for example, a promoter, operably linked to the ORF.

Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, pSVL (Pharmacia).

The nucleic acid sequences of the invention are further directed to sequences which encode variants of the described nucleic acids. These amino acid sequence variants may be prepared by methods known in the art by introducing appropriate nucleotide changes into a native or variant polynucleotide. There are two variables in the construction of amino acid sequence variants: the location of the mutation and the nature of the mutation. The amino acid sequence variants of the nucleic acids are preferably constructed by mutating the polynucleotide to give an amino acid sequence that does not occur in nature. These amino acid alterations can be made at sites that differ in the nucleic acids from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site. Amino acid sequence deletions generally range from about 1 to 30 residues, preferably about 1 to 10 residues, and are typically contiguous. Amino acid insertions include amino- and/or carboxy-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells.

In a preferred method, polynucleotides encoding the novel nucleic acids are changed via site-directed mutagenesis. This method uses oligonucleotide sequences that encode the polynucleotide sequence of the desired amino acid variant, as well as a sufficient adjacent nucleotide on both sides of the changed amino acid to form a stable duplex on either side of the site of being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982). PCR may also be used to create amino acid sequence variants of the novel nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives the desired amino acid variant.

A further technique for generating amino acid variants is the cassette mutagenesis technique described in Wells et al, Gene 34:315 (1985); and other mutagenesis techniques well known in the art, such as, for example, the techniques in Sambrook et al., supra, and Current Protocols in Molecular Biolog, Ausubel et al., supra Due to the inherent degeneracy of the genetic code, other DNA sequences which encode substantially the same or a functionally equivalent amino acid sequence may be used in the practice of the invention for the cloning and expression of these novel nucleic acids. Such DNA sequences include those which are capable of hybridizing to the appropriate novel nucleic acid sequence under stringent conditions.

### HOSTS

The present invention further provides host cells genetically engineered to contain the polynucleotides of the invention. For example, such host cells may contain nucleic acids of the invention introduced into the host cell using known transformation, transfection or infection methods. The present invention still further provides host cells genetically engineered to express the polynucleotides of the invention, wherein such polynucleotides are in operative association with a regulatory sequence heterologous to the host cell which drives expression of the polynucleotides in the cell.

The host cell can be a higher eukaryotic host cell, such as a mammalian cell or an insect cell, a lower eukaryotic host cell, such as a yeast cell, or the host cell can be a prokaryotic cell, such as a bacterial cell. Introduction of the recombinant construct into the host cell can be effected by calcium phosphate transfection, DEAE, dextran mediated transfection, or electroporation (Davis, L. et al., Basic Methods in Molecular Biology (1986)). The host cells containing one of polynucleotides of the invention, can be used in conventional manners to produce the gene product encoded by the isolated fragment (in the case of an ORF) or can be used to produce a heterologous protein under the control of the EMF.

Any host/vector system can be used to express one or more of the ORFs of the present invention. These include, but are not limited to, eukaryotic hosts such as HeLa cells, Cv-1 cell, COS cells, and Sf9 cells, as well as prokaryotic host such as E. coli and B. subtilis. The most preferred cells are those which do not normally express the particular polypeptide or protein or which expresses the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., in Molecular Cloning: A Laboratory Manual, Second Edition, Cold Spring Harbor, New York (1989), the disclosure of which is hereby incorporated by reference.

### POLYPEPTIDES OF THE INVENTION

The isolated polypeptides of the invention include, but are not limited to, a polypeptide comprising the amino acid sequence of SEQ ID NO: 2. The polypeptides of the invention further include polypeptides which comprise one or more specific domains of the amino acid sequence of SEQ ID NO: 2.

Protein compositions of the present invention may further comprise an acceptable carrier, such as a hydrophilic, e.g., pharmaceutically acceptable, carrier.

The invention also relates to methods for producing a polypeptide comprising growing a culture of the cells of the invention in a suitable culture medium, and purifying the protein from the culture. For example, the methods of the invention include a process for producing a polypeptide in which a host cell containing a suitable expression vector that includes a polynucleotide of the invention is cultured under conditions that allow expression of the encoded polypeptide. The polypeptide can be recovered from the culture, conveniently from the culture medium, and further purified. Preferred embodiments include those in which the protein produced by such process is a full length or mature form of the protein.

The invention further provides a polypeptide including an amino acid sequence that is substantially equivalent to SEQ ID NO: 2. Polypeptides according to the invention can have at least about 98%, and more typically at least about 99%, and even more typically 99.5 sequence identity to SEQ ID NO: 2.

The present invention further provides isolated polypeptides encoded by the nucleic acid fragments of the present invention or by degenerate variants of the nucleic acid fragments of the present invention. By “degenerate variant” is intended nucleotide fragments which differ from a nucleic acid fragment of the present invention (e.g., an ORF) by nucleotide sequence but, due to the degeneracy of the genetic code, encode an identical polypeptide sequence. Preferred nucleic acid fragments of the present invention are the ORFs that encode proteins. A variety of methodologies known in the art can be utilized to obtain any one of the isolated polypeptides or proteins of the present invention. At the simplest level, the amino acid sequence can be synthesized using commercially available peptide synthesizers. This is particularly useful in producing small peptides and fragments of larger polypeptides. Fragments are useful, for example, in generating antibodies against the native polypeptide. In an alternative method, the polypeptide or protein is purified from cells which naturally produce the polypeptide or protein. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual (supra); Ausubel et al, Current Protocols in Molecular Biology (supra).

The polypeptides and proteins of the present invention can alternatively be purified from cells which have been altered to express the desired polypeptide or protein. One skilled in the art can readily adapt procedures for introducing and expressing either recombinant or synthetic sequences into eukaryotic or prokaryotic cells in order to generate a cell which produces one of the polypeptides or proteins of the present invention. The purified polypeptides can be used in in vitro binding assays which are well known in the art to identify molecules which bind to the polypeptides.

The protein may also be produced by known conventional chemical synthesis. Methods for constructing the proteins of the present invention by synthetic means are known to those skilled in the art. For polypeptides more than about 100 amino acid residues, a number of smaller peptides will be chemically synthesized and ligated either chemically or enzymatically to provide the desired full-length polypeptide. The synthetically-constructed protein sequences, by virtue of sharing primary, secondary or tertiary structural and/or conformational characteristics with proteins may possess biological properties in common therewith. Thus, they may be employed as biologically active or immunological substitutes for natural, purified proteins in screening of therapeutic compounds and in immunological processes for the development of antibodies.

The proteins provided herein also include proteins characterized by amino acid sequences substantially equivalent to those of purified proteins but into which modification are naturally provided or deliberately engineered. For example, modifications in the peptide or DNA sequences can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein.

Other fragments and derivatives of the sequences of proteins which would be expected to retain protein activity in whole or in part and may thus be useful for screening or other immunological methodologies may also be easily made by those skilled in the art given the disclosures herein. Such modifications are intended to be encompassed by the present invention.

The protein of the invention may also be expressed in a form that will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLab (Beverly, Mass.), Pharmacia (Piscataway, N.J.) and Invitrogen (Carlsbad, Calif.), respectively. The protein also can be tagged with an epitope and subsequently purified by using a specific antibody directed to such epitope. One such epitope (“Flag”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, also can be employed to provide a substantially homogeneous isolated recombinant protein. The protein thus purified is substantially free of other mammalian proteins and is defined in accordance with the present invention as an “isolated protein.

The polypeptide sequences encoded by SEQ ID NO: 1 have numerous applications in techniques known to those skilled in the art of molecular biology. The polypeptides of the invention are related to JNK interacting protein of rat (Genbank Accession No. AF092450) and mouse (Genbank Accession No. AF05461 1). The polypeptides of the invention and/or their agonists and antagonists are useful in methods for inhibiting diseased states associated with the response to oxidative stress. The polypeptides of the invention may also be used to generate antibodies for diagnosis or therapy of such disease states.

### USES AND BIOLOGICAL ACTIVITY

The polynucleotides and proteins of the present invention exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

### RESEARCH USES AND UTILITIES

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The proteins provided by the present invention can similarly be used in assays to determine biological activity, including in a panel of multiple proteins for high-throughput screening; to raise antibodies or to elicit another immune response; as a reagent (including a labeled reagent) in assays designed to determine quantitative levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation Molecular Cloning: A Laboratory Manual, 2d ed., Cold Spring Harbor Laboratory Press, Sambrook, J., E. F. Fritsch and T. Maniatis eds., 1989, and Methods in Enzymology: Guide to Molecular Cloning Techniques, Academic Press, Berger, S. L. and A. R. Kimmel eds., 1987.

### PHARMACEUTICAL FORMULATIONS AND ROUTES OF ADMINISTRATION

A protein of the present invention (from recombinant or non-recombinant sources) may be administered to a patient in need, by itself, or in pharmaceutical compositions where it is mixed with suitable carriers or excipient(s) at doses to treat or ameliorate a variety of disorders. Techniques for formulation and administration of the compounds of the instant application may be found in Remington's Pharmaceutical Sciences, Mack Publishing Co., Easton, Pa., latest edition. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing the method of treatment or use of the present invention, a therapeutically effective amount of protein of the present invention is administered to a mammal having a condition resulting in oxidative stress to be treated. Such conditions include stroke and Alzheimer's disease, Parkinson's disease and amyotrophic lateral sclerosis (Lou Gehrig's disease). Protein of the present invention may be administered in accordance with the method of the invention either alone or in combination with other therapies.

### ROUTES OF ADMINISTRATION

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of protein of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

Furthermore, one may administer the drug in a targeted drug delivery system, for example, in a liposome coated with a specific antibody, targeting, for example, neuronal tissue. The liposomes will be targeted to and taken up selectively by the afflicted tissue.

### GENE THERAPY

Polynucleotides of the present invention can also be used for gene therapy for the treatment of disorders which are mediated by hJIP-1/IB1. Such therapy would achieve its therapeutic effect by introduction of the appropriate hJIP-1/IB1 polynucleotide (e.g., SEQ ID NO: 1) which contains ahJIP-1/IB 1gene (sense), into cells of subjects having the oxidative stress disorder. Delivery of sense hJIP-1/IB1 polynucleotide constructs can be achieved using a recombinant expression vector such as a chimeric virus or a colloidal dispersion system. An expression vector including the hJIP-1/IB1 polynucleotide sequence could be introduced to the subject's cells ex vivo after removing, for example, stem cells from a subject's bone marrow. The cells are then reintroduced into the subject, (e.g., into subject's bone marrow).

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), Rous Sarcoma Virus (RSV), and gibbon ape leukemia virus (GaLV), which provides a broader host range than many of the murine viruses. A number of additional retroviral vectors can incorporate multiple genes. All of these vectors can transfer or incorporate a gene for a selectable marker so that transduced cells can be identified and selected for. By inserting a hJIP-1/IB1 sequence of interest into the viral vector, along with another gene which encodes the ligand for a receptor on a specific target cell, for example, the vector is now target specific. Preferred targeting is accomplished by using an antibody to target the retroviral vector. Those of skill in the art will know of, or can readily ascertain without undue experimentation, specific polynucleotide sequences which can be inserted into the retroviral genome to allow target specific retroviral vector containing the hJIP-1/IB1 sense or antisense polynucleotide.

Since recombinant retroviruses are defective, they require assistance to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsidation. Helper cell lines which have deletions of the packaging signal include but are not limited to .PSI.2, PA317 and PA12, for example. These cell lines produce empty virions, since no genome is packaged. If a retroviral vector in which the packaging signal is intact, but the structural genes are replaced by other genes of interest is introduced into such cells, the vector will be packaged and vector virions produced.

Another targeted delivery system for hJIP-1/IB1 polynucleotide is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. For a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The surface of the targeted delivery system may be modified in a variety of ways. In the case of a liposomal targeted delivery system, lipid groups can be incorporated into the lipid bilayer of the liposome to maintain the targeting ligand in stable association with the liposomal bilayer. Various linking groups can be used for joining the lipid chains to the targeting ligand.

In general, the compounds bound to the surface of the targeted delivery system will be ligands and receptors which will allow the targeted delivery system to find and “home in” on the desired cells. A ligand may be any compound of interest which will bind to another compound, such as a receptor. In general, surface membrane proteins which bind to specific effector molecules are referred to as receptors. In the present invention, antibodies are preferred receptors. Antibodies can be used to target liposomes to specific cell-surface ligands. Preferred cell-surface ligands are those that are selectively expressed on neuronal tissues.

### ANTIBODIES

Another aspect of the invention is an antibody that specifically binds the polypeptide of the invention. Such antibodies can be either monoclonal or polyclonal antibodies, as well as fragments thereof and humanized forms or fully human forms, such as those produced in transgenic animals. The invention further provides a hybridoma that produces an antibody according to the invention. Antibodies of the invention are useful for detection and/or purification of the polypeptides of the invention.

Protein of the invention may also be used to immunize animals to obtain polyclonal and monoclonal antibodies that react specifically with the protein. Such antibodies may be obtained using either the entire protein or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides are known in the art, for example, as in R. P. Merrifield, J. Amer. Chem. Soc. 85, 2149-2154 (1963); J. L. Krstenansky, et al., FEBS Lett. 211, 10 (1987). Monoclonal antibodies binding to the protein of the invention may be useful diagnostic agents for the immunodetection of the protein. Neutralizing monoclonal antibodies binding to the protein may also be useful therapeutics for conditions associated with excess production or accumulation of the protein. In general, techniques for preparing polyclonal and monoclonal antibodies as well as hybridomas capable of producing the desired antibody are well known in the art (Campbell, A. M., Monoclonal Antibodies Technology: Laboratory Techniques in Biochemistry and Molecular Biology, Elsevier Science Publishers, Amsterdam, The Netherlands (1984); St. Groth et al., J. Immunol. 35:1-21(1990); Kohler and Milstein, Nature 256:495-497 (1975)), the trioma technique, the human B-cell hybridoma technique (Kozbor et al., Immunology Today 4:72 (1983); Cole etal., in Monoclonal Antibodies and Cancer Therapy, Alan R. Liss, Inc. (1985), pp. 77-96).

Any animal (mouse, rabbit, etc.) which is known to produce antibodies can be immunized with a peptide or polypeptide of the invention. Methods for immunization are well known in the art. Such methods include subcutaneous or intraperitoneal injection of the polypeptide. One skilled in the art will recognize that the amount of the protein encoded by the ORF of the present invention used for immunization will vary based on the animal which is immunized, the antigenicity of the peptide and the site of injection. The protein that is used as an immunogen may be modified or administered with an adjuvant to increase the protein's antigenicity. Methods of increasing the antigenicity of a protein are well known in the art and include, but are not limited to, coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Research, 175:109-124 (1988)).

Hybridomas secreting the desired antibodies are cloned and the class and subclass is determined using procedures known in the art (Campbell, A. M., Monoclonal Antibody Technology: Laboratory Techniques in Biochemistry and Molecular Biology, Elsevier Science Publishers, Amsterdam, The Netherlands (1984)). Techniques described for the production of single chain antibodies (U.S. Pat. No. 4,946,778) can be adapted to produce single chain antibodies to proteins of the present invention.

For polyclonal antibodies, antibody containing antiserum is isolated from the immunized animal and is screened for the presence of antibodies with the desired specificity using one of the above-described procedures. The present invention farther provides the above-described antibodies in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, etc. Procedures for accomplishing such labeling are well-known in the art, for example, see (Stemberger, L. A. et al., J. Histochem. Cytochem. 18:315 (1970); Bayer, E. A. et al., Meth. Enzym. 62:308 (1979); Engval, E. et al, Immunol. 109:129 (1972); Goding, J. W. J. Immunol. Meth. 13:215 (1976)).

The labeled antibodies of the present invention can be used for in vitro, in vivo, and in situ assays to identify cells or tissues in which a fragment of the polypeptide of interest is expressed. The antibodies also may be used directly in therapies or other diagnostics. The present invention further provides the above-described antibodies immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir, D. M. et al., Handbook of Experimental Inmunology 4th Ed., Blackwell Scientific Publications, Oxford, England, Chapter 10 (1986); Jacoby, W. D. et al., Meth. Enzym. 34 Academic Press, N.Y. (1974)). The immobilized antibodies of the present invention can be used for in vitro, in vivo, and in situ assays as well as for immuno-affinity purification of the proteins of the present invention.

### USE OF NUCLEIC ACIDS AS PROBES

Another aspect of the subject invention is to provide for polypeptide-specific nucleic acid hybridization probes capable of hybridizing with naturally-occurring nucleotide sequences. The hybridization probes of the subject invention may be derived from the nucleotide sequence of SEQ ID NO: 1. Because the corresponding gene is only expressed in a limited number of tissues, especially adult tissues, a hybridization probe derived from SEQ ID NO: 1 can be used as an indicator of the presence of RNA of cell type of such a tissue in a sample as shown in Example 5.

Any suitable hybridization technique can be employed, such as, for example, in situ hybridization-PCR as described by U.S. Pat. Nos. 4,683,195 and 4,965,188 which provide additional uses for oligonucleotides based upon the nucleotide sequences. Such probes used in PCR may be of recombinant origin, may be chemically synthesized, or a mixture of both. The probe will comprise a discrete nucleotide sequence for the detection of identical sequences or a degenerate pool of possible sequences for identification of closely related genomic sequences.

Other means for producing specific hybridization probes for nucleic acids include the cloning of nucleic acid sequences into vectors for the production of MRNA probes. Such vectors are known in the art and are commercially available and may be used to synthesize RNA probes in vitro by means of the addition of the appropriate RNA polymerase as T7 or SP6 RNA polymerase and the appropriate radioactively labeled nucleotides. The nucleotide sequences may be used to construct hybridization probes for mapping their respective genomic sequences. The nucleotide sequence provided herein may be mapped to a chromosome or specific regions of a chromosome using well known genetic and/or chromosomal mapping techniques. These techniques include in situ hybridization, linkage analysis against known chromosomal markers, hybridization screening with libraries or flow-sorted chromosomal preparations specific to known chromosomes, and the like. The technique of fluorescent in situ hybridization of chromosome spreads has been described, among other places, in Verma et al. (1988) Human Chromosomes: A Manual of Basic Techniques, Pergamon Press, New York NY.

Fluorescent in situ hybridization of chromosomal preparations and other physical chromosome mapping techniques may be correlated with additional genetic map data. Examples of genetic map data can be found in the 1994 Genome Issue of Science (265:1981 f). Correlation between the location of a nucleic acid on a physical chromosomal map and a specific disease (or predisposition to a specific disease) may help delimit the region of DNA associated with that genetic disease. The nucleotide sequences of the subject invention may be used to detect differences in gene sequences between normal, carrier or affected individuals. The nucleotide sequence may be used to produce purified polypeptides using well known methods of recombinant DNA technology. Among the many publications that teach methods for the expression of genes after they have been isolated is Goeddel (1990) Gene Expression Technology, Methods and Enzymology, Vol. 185, Academic Press, San Diego. Polypeptides may be expressed in a variety of host cells, either prokaryotic or eukaryotic. Host cells may be from the same species from which a particular polypeptide nucleotide sequence was isolated or from a different species. Advantages of producing polypeptides by recombinant DNA technology include obtaining adequate amounts of the protein for purification and the availability of simplified purification procedures.

The present invention is illustrated in the following examples. Upon consideration of the present disclosure, one of skill in the art will appreciate that many other embodiments and variations may be made in the scope of the present invention. Accordingly, it is intended that the broader aspects of the present invention not be limited to the disclosure of the following examples.

### EXAMPLES

### EXAMPLE 1

### Cloning hJIP-1/IB1

Clone 2.5 was isolated using a yeast two hybrid screen (Zhang et al., 1998). The isolated clone was sequenced and corresponds to nucleotides 226-2836 of SEQ ID NO: 1. The 5′ region of hJIP-1/IB1 was identified by PCR of a human cDNA library (Human Brain Hippocampus Marathon-Ready cDNA, Clontech, Inc.).

### EXAMPLE 2

### Expression of hJIP-1/IB1-GST Fusions

Clone 2.5, nucleotides 226-2836 of SEQ ID NO: 1, was subcloned into EcoRI and Xhol site of the pGEX-4T-2 (Pharmacia) expression vector in frame with a glutathione S-transferase moiety. The resulting fusion protein (GST-2.5) was expressed and purified from E. coli host DH5α (Life Technologies) using standard methods for purifying GST-fusion proteins.

### EXAMPLE 3

### Inhibition by hJIP-1/IB1 of c-Jun Phosphorylation by JNK3

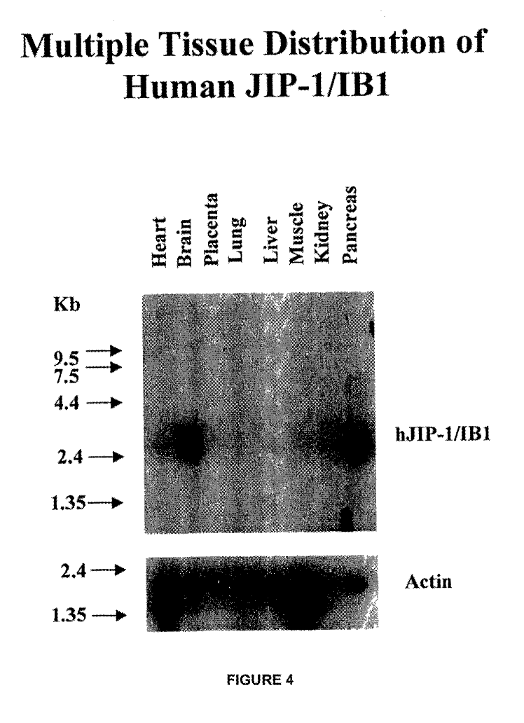

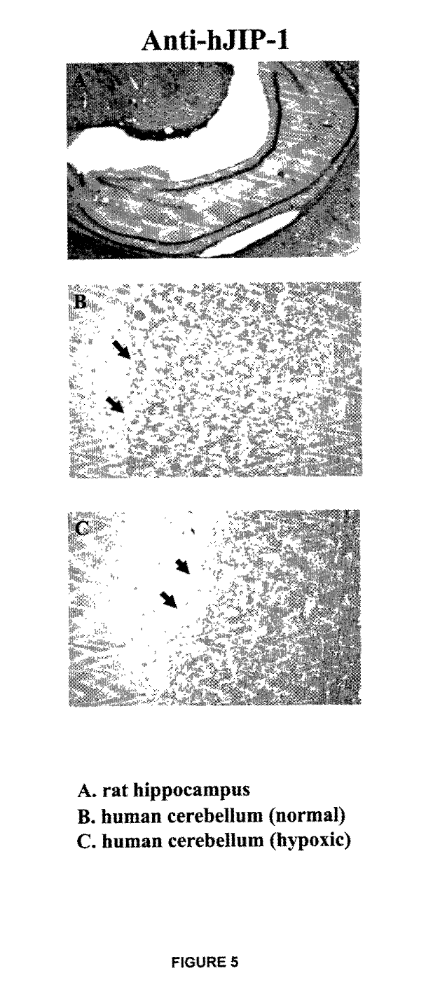

This example shows that hJIP1/IB1, in particular that region corresponding to amino acid residues 76-211 of SEQ ID NO: 2, both is phosphorylated by the neuron-specific c-Jun amino terminal kinase (JNK3) and inhibits c-Jun phosphorylation by JNK3. In vitro phosphorylation assays were performed according to the method of Dickens et al. (1997) using a kinase assay (c-Jun N-terminal Assay Kit, Stratagene, Inc.). As shown in FIG. 4, this protein greatly reduces phosphorylation of a GST c-Jun fusion protein in a concentration dependent manner. FIG. 5 also shows that hJIP1/IB1 is a substrate for JNK3 in the same in vitro phosphorylation assay.

### EXAMPLE 4

### Generating hJIP-1/IB1 Polyclonal Antisera

A 19 amino acid residue peptide Gly Val Lys Ile Gly Val Lys Ala Asp Asp Ser Gln Glu Ala Lys Gly Asn Lys Cys (SEQ ID NO: 3) was synthesized corresponding to amino acid residues 617-635 of hJIP-1/IB1 (SEQ ID NO: 2). Rabbits were immunized with the synthetic peptide according to standard procedures to produce high-titer, polyclonal antisera. Specificity of this antisera was verified immunocytochemically on human and rat CNS tissues and by immunoprecipitation of hJIP1/IB1 from mouse neuroblastoma clonal cultures (N₂A cells, ATCC).

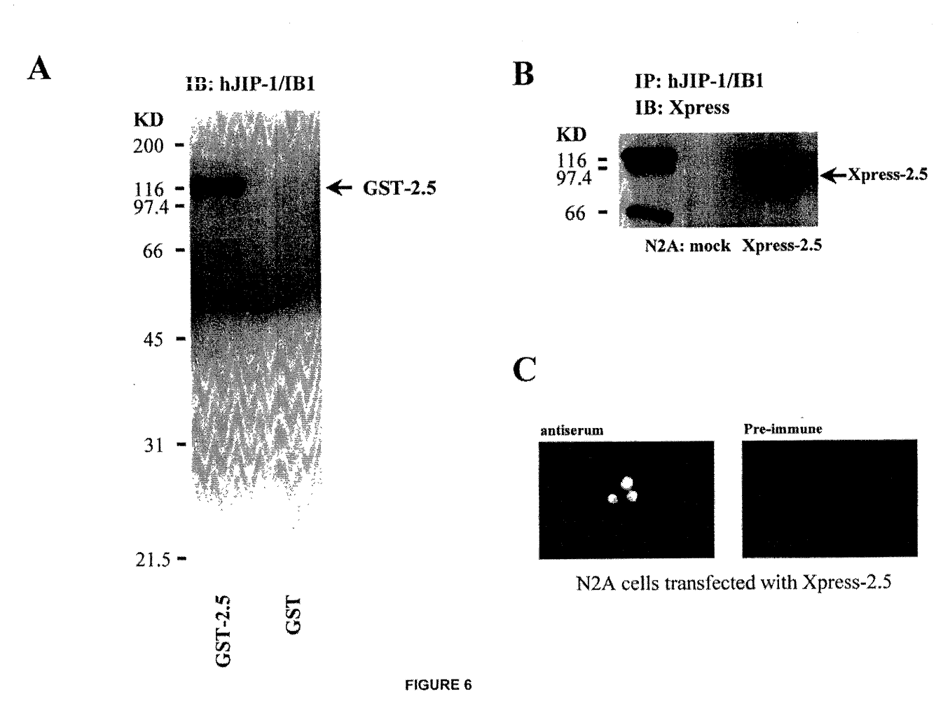

Anti-hJIP1/IB1 specificity was confirmed in three ways: by immunoblotting (Western blotting) purified protein (FIG. 6A), by immunoprecipitating and Western blotting N₂A cells transfected with Xpress tagged clone 2.5 (FIG. 6B), and by immunohistochemical staining N₂A cells transfected with Xpress tagged clone 2.5 (FIG. 6C). Equal amounts of GST-2.5 and GST were subjected to SDS-PAGE. Immunoblotting using anti-hJIP-1/IB1 showed the antibody only recognizes GST-2.5. N2A cells were transfected with Xpress-2.5 cDNA. The cell lysate was immunoprecipitated with anti hJIP-1/IB1 following SDS-PAGE. The immunoprecipitate was recognized by anti-Xpress. N2A cells were transfected with Xpress-2.5 cDNA. Antiserum of hJIP-1/IB1 recognized transfected cells, but preimmune antiserum could not.

### EXAMPLE 5

### Immunocytochemistry

In normal human CNS tissues HJIP1/IB1 is diffusely localized to neuronal cytoplasm and concentrated focally near the nuclear envelope. One possibility is that it may serve as a “road-block” near nuclear pores preventing entry of JNKs and c-Jun into the nucleus and triggering the apoptosis cascade. As shown in Table 1, hJIPl/IBl is most strongly expressed in the CA2 and CA3 regions of normal hippocampus and less so in CA1 and CA4. In the cerebellum, Purkinje cells are most strongly stained. Acute hypoxic changes, histologically confirmed in these regions by hematoxylin and eosin stains of “red” or eosinophilic neurons, are correlated with reduced anti-hJIP1/IB1 labeling of CA1 pyramidal neurons which contrasts with sustained staining of CA2 and CA3 neurons.

TABLE 1
Human JIP-1/IB1 immunoreactivity in selected human brain sections.
Immunohistochemistry reveals most intense staining of CA2, and
CA4 regions in hippocampus and cerebellar Purkinje cells in normal
controls. With acute hypoxia, CA1 shows major loss of staining in CA1,
subiculum and Purkinje cells. Slight reduction of staining is seen in these
regions in patients with clinical and neuropathological findings of AD and
chronic hypoxia. There is also intranuclear staining in patients with AD
and acute and chronic hypoxic changes.

| | |
|---|---|
| HIPPOCAMPUS | CEREBELLUM |

| | | | | | | Purkinje | White |
|---|---|---|---|---|---|---|---|
| | CA4 | CA3 | CA2 | CA1 | Subiculum | Cells | Matter |
| Control | ++ | +++ | +++ | + | + | ++ | ++ |
| (# 197) | | | | | | (C) | |
| AD | ++ | ++ | ++ | + | + | ++ | ++ |
| (# 458) | | | | | | (C/N) | |
| Acute | + | ++ | ++ | − | − | − | ++ |
| Hypoxia | | | | | | | |
| (# 187) | | | | | | | |
| Control | ++ | +++ | +++ | + | + | ++ | ++ |
| (# 521) | | | | | | (C) | |
| AD | + | +++ | +++ | + | + | ++ | ++ |
| (# 502) | | | | | | (C/N) | |
| Chronic | ++ | ++ | ++ | + | + | ++ | + |
| Hypoxia | | | | | | (C/N) | |
| (# 378) | | | | | | | |

− = negligible expression
+ = low expression
++ = moderate expression
+++ = high expression
N = Nuclear expression
C = Cytoplasmic expression
C/N = Cytoplasmic and Nuclear expression

### EXAMPLE 6

### Northern Analysis—Tissue Distribution

Northern analysis of multiple tissue mRNAs reveals hJIP1/IB1 is strongly expressed in brain and pancreas (FIG. 4). Clone 2.5 was used as a probe as described in Zhang et al. (1998).

### EXAMPLE 7

### Neuronal Model Systems

Selective vulnerability of neuronal model systems. Two experimental systems demonstrate an inverse relationship of hJIP1/IB1 to JNK3 activation. Rat organotypic hippocampal cultures and clonal neuroblastoma cultures were exposed to short-term hypoxia with and without re-oxygenation.

The same neuronal distribution of hJIP1/IB1 as in human CNS is seen in the normal rat hippocampus (FIG. 5A) and in the organotypic cultures (not shown). In human cerebellum there is loss of cytoplasmic immunoreactivity of Purkinje cells under acute hypoxic conditions compared to normal (FIGS. 5B, C). Under chronic stress in both hypoxic and in AD patients, there is loss of hJIP1/IB1 staining of Purkinje cells (Table 1).

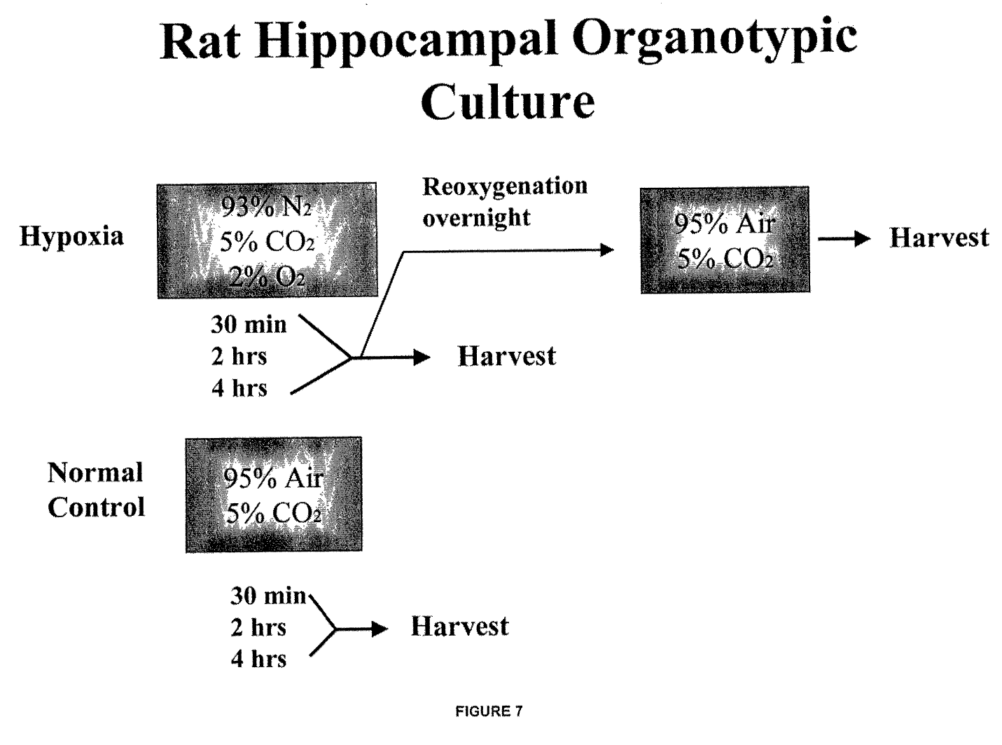

The experimental paradigm for rat organotypic cultures is shown in FIG. 7.

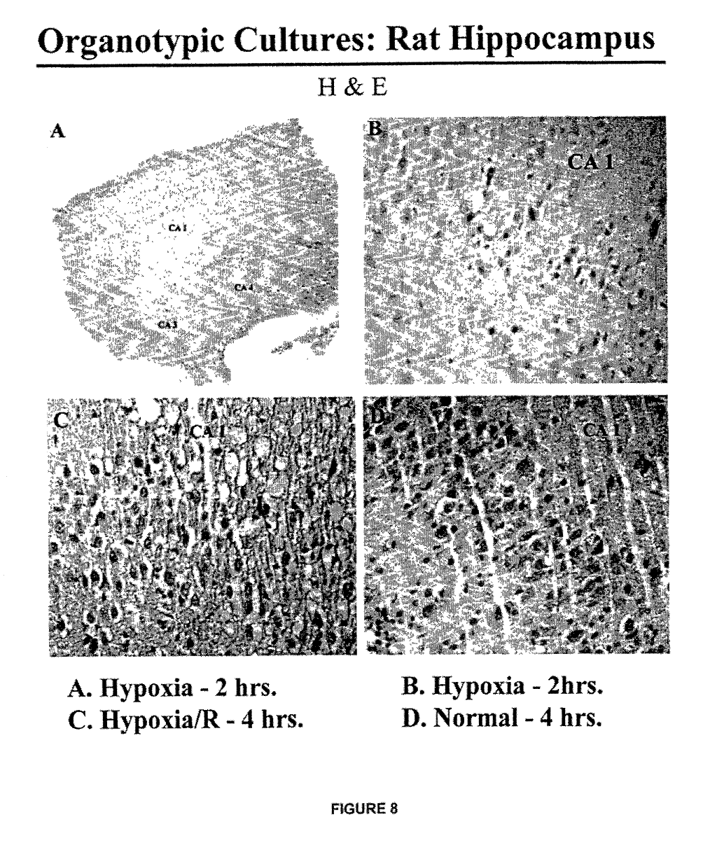

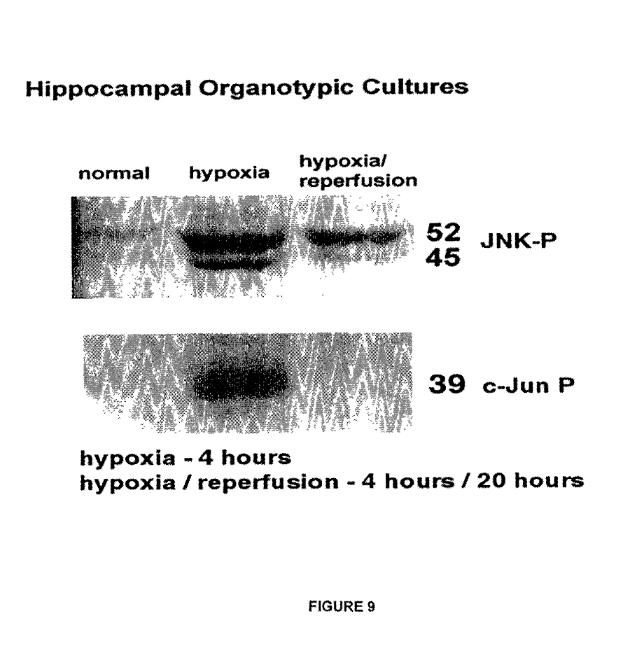

Included, is “re-oxygenation,” the addition to cultures of glucose-containing medium under normal, ambient atmospheric conditions. By 2 hrs. post-hypoxia, neuronal eosinophilia and edema are present in the CA1 of the hippocampus (FIG. 8; Table 2). Cultures also show apoptosis (Table 3) as determined by the presence of single stranded DNA (ssDNA) and a positive signal for the ApopTag, and decreased anti-hJlP1/IB1 immunostaining and nuclear translocation of DENN/MADD, especially in CA1. Neuronal apoptosis is markedly and rapidly induced by 4 hours hypoxia/reoxygenation (Table 3). In FIG. 9, immunoblots show hypoxia with or without re-oxygenation increased activation of JNKs by 4 hours. C-Jun is activated (phosphorylation of serine 63), detected by anti-phospho-c-Jun, after 4 hours hypoxia alone (FIG. 9).

TABLE 2
Rat Hippocampal Culture. Anti-clone 2.5 (hJIP1/IB1) staining shows
decreased reactivity in CA1 region of the hippocampus after 2 hrs.
hypoxia. After 4 hrs. hypoxia plus reperfasion, there is more extensive
loss (CA1-3) plus nuclear translocation of anti-DENN/MADD staining
comparable to that reported in the hypoxic post-mortem human CNS
(Zhang et al., 1998).

| | | ANTI-hJIP-1/1B1 | ANTI-DENN/MADD |
|---|---|---|---|
| 30 min | Normal | No change | cytoplasm |
| | Hypoxia | No change | cytoplasm |
| | Hypoxia/R | No | cytoplasm |
| 2 hrs | Normal | No change | cytoplasm |
| | Hypoxia | Decreased (CA1) | cytoplasm |
| | Hypoxia/R | No change | cytoplasm |
| 4 hrs | Normal | No change | cytoplasm |
| | Hypoxia | Decreased (CA1) | cytoplasm |
| | Hypoxia/R | Decreased (CA1-3) | +nucleolus |

To test the hJIP-1/IB1 response to oxidant stress, rat hippocampal organotypic cultures were rendered hypoxic with or without reperfusion, resulting in apoptosis in the CA-1 region accompanied by a sustained decrease of hJIP-1/IB1 expression and increased JNK activation. In controls, the strongest expression of hJIPl/IB is consistently seen in the CA2 and CA3 regions in contrast to the CA1 region; these areas are not selectively vulnerable to oxidant stresses. The normal distribution of JNK and hJIP1/IB1 and the pattern of responses to hypoxia mirrors that in the CA1 region of the human hippocampus, a site normally enriched in JNK3, but expressing relatively less hJIP1/IB1 than do the CA2 and CA3 regions. These in vivo findings suggest an inverse relationship of these two interactants: the greater the ratio of hJIP1/IB1 to JNK3, the higher the protective threshold of the neurons. Potential applications of hJIP1/IB1 to inhibit or modulate c-Jun mediated apoptosis is strongly suggested by our results in the human brain, and in experimental model systems.

TABLE 3
Rat Hippocampal Culture. After 2 hrs. hypoxia, there is eosinophilia and
edema in the CA1 region. Anti-ssDNA immunostain shows positive cells
in the same region indicative of apoptosis. These changes are extensive
after 4 hrs. hypoxia, especially when followed by reperfusion.

| | |
|---|---|
| H&E | Anti |

| | | Eosinophilia | Edema | Apoptag | ssDNA |
|---|---|---|---|---|---|
| 30 min | Normal | NA | NA | − | − |
| | Hypoxia | − | − | − | − |
| | Hypoxia | − | − | − | − |
| 2 hrs | Normal | − | − | | − |
| | Hypoxia | +++ (CA1) | +++ (CA1) | − | + (CA1) |
| | Hypoxia/R | + | ++ | − | − |
| 4 hrs | Normal | − | − | − | − |
| | Hypoxia | ++ | ++ | − | − |
| | Hypoxia/R | +++ | ++++ | ++ | +++ |
| | | (CA1-3) | (CA1-3) | (CA1) | (CA1) |

All references cited within the body of the instant specification are hereby incorporated by reference in their entirety.

While the invention has been described and illustrated with reference to certain particular embodiments thereof, those skilled in the art will appreciate that various changes, modifications and substitutions cab be made therein without departing from the spirit and scope of the invention as set forth in the claims that follow, which should be interpreted as broadly as reasonable.

### REFERENCES

1) Bonney C, Nicod P and Waeber G. (1998) IB1, a JIP-1 related nuclear protein present in insulin secreting cells. J. Biol Chem 273:1843-46.

2) Dickens M, Rogers J S, Cavanagh J, Raitano A, Xia Z, Halpern J R, Greenburg M E, Sawyers C L and Davis R J. (1997) A cytoplasmic inhibitor of the JNK signal transduction pathway. Science 277:693-96.

3) Elion R A. (1988) Routing MAP kinase cascade. Science 281:1625-26.

4) Herdegen T, Claret F X, Kallunki T, Martin-Villalla A, Winter C, Hunter T, Karen M. (1998) Lasting N-terminal phosphorylation of c-Jun and activation of c-Jun N-terminal kinases after neuronal injury. J Neurosci 18:5124-35.

5) Kyriakis J, Avruch J. (1996) Protein kinase cascades activated by stress and inflammatory cytokines. BioEssays 18:567-77.

6) Mohit A A, Martin J, Miller C A. (1995) p49^{3F12} kinase: A novel MAP kinase expressed in a subset of neurons in the human nervous system. Neuron 14:67-78.

7) Mooser V, Maillard A, Bonny C, Steinmann M, Shaw P, Yamall D P, Bums D K, Schorderet D F, Nicod P, Waeber G. (1999) Genomic organization, fine-mapping and expression of the human iset-brain 1(1B 1)/c-Jun-amino-terminal kinase interacting protein-1 (JIP1) gene. Genomics 55:202-208.

8) Zhang Y, Zhou L, Miller C A. (1998) A splicing variant of a death domain protein that is regulated by a mitogen-activated kinase is a substrate for c-Jun N-terminal kinase in the human central nervous system. PNAS 95:2586-91.

What is claimed is:

1. A method of treating a neurological disorder in a human patient which comprises administering to said human patient an effective amount of a composition comprising a polypeptide comprising a sequence substantially equivalent to SEQ ID NO: 2.

2. The method of claim 1 wherein the composition further comprises a pharmaceutically acceptable carrier.

3. The method of claim 1 wherein the composition is administered orally, transdermally, intravenously, intrasynovially, intramuscularly, intraocularly, intranasally, intrathecally, or topically.

4. The method of claim 1 wherein administering the composition is in conjunction with another method of treating said neurological disorder.

5. The method of claim 1, wherein the neurological disorder is caused by oxidative stress response in neuronal tissue.

6. The method of claim 1, wherein the neurological disorder is caused by the activation of a neuron specific, stress-activated protein kinase.

7. The method of claim 6, wherein the protein kinase is c-Jun amino-terminal kinase 3.

8. The method of claim 1 wherein the neurological disorder is a disorder selected from dementia, dementia of the Alzheimer's type, bipolar disorders, mood disorder with depressive features, mood disorder with major depressive-like episode, mood disorder with manic features, mood disorder with mixed features, substance-induced mood disorder and mood disorder not otherwise specified (NOS), panic disorder without agoraphobia, panic disorder with agoraphobia, agorathobia without history of panic disorder, social phobia, postraumatic stress disorder, acute stress disorder, substance-induced anxiety disorder and anxiety disorder not otherwise specified (NOS), dyskinesias and behavioral manifestations of mental retardation, conduct disorder and autistic disorder.

9. The method of claim 8, wherein dementia is selected from the group consisting of vascular dementia, dementia due to HIV disease, dementia due to head trauma, dementia due to Parkinson's disease, dementia due to Huntington's disease, dementia due to Pick's disease, dementia due to Creutzfeldt-Jakob disease, substance-induced persisting dementia, dementia due to multiple etiologies and dementia not otherwise specified (NOS).

10. The method of claim 8, wherein said dementia is dementia of the Alzheimer's type.

11. The method of claim 10, wherein dementia of the Alzheimer's type is selected from the group consisting of dementia of the Alzheimer's type with early onset uncomplicated, dementia of the Alzheimer's type with early onset with delusions, dementia of the Alzheimer's type with early onset with depressed mood, dementia of the Alzheimer's type with late onset uncomplicated, dementia of the Alzheimer's type with late onset with delusions and dementia of the Alzheimer's type with late onset with depressed mood.

12. The method of claim 1, wherein the composition is administered in a targeted drug delivery system.

13. The method of claim 12, wherein the targeted drug delivery system is a liposome coated with an antibody that specifically targets neuronal tissue.

14. A method of treating Alzheimer's disease, stroke, amyotrophic lateral sclerosis, age associated memory impairment or Parkinson's disease in a human subject, the method comprising administering to said human an effective amount of a composition comprising a polynucleotide having a sequence that is substantially equivalent to SEQ ID NO: 1.

15. The method of claim 14, wherein the composition is administered to the subject's cells using a recmobinant expression vector that comprises a sequence substantially equivalent to SEQ ID NO: 1.

16. The method of claim 15, wherein administering the composition further comprises:
removing stem cells from a subject's bone marrow;
introducing the recombinant expression vector into the removed stem cells; and
re-introducing the stem cells into the subject's bone marrow.

17. A method of treating a neurological disease in a human subject selected from the group consisting of Alzheimer's disease, stroke, amyotrophic lateral sclerosis, age associated memory impairment and Parkinson's disease, the method comprising administering to said human an effective amount of a composition comprising a polypeptide having a sequence that is substantially equivalent to SEQ ID NO: 2.

18. The method of claim 17 wherein the composition further comprises a pharmaceutically acceptable carrier.

19. The method of claim 17 wherein the composition is administered orally, transdermally, intravenously, intrasynovially, intramuscularly, intraocularly, intranasally, intrathecally, or topically.

20. The method of claim 17 wherein the method is used in conjunction with another method of treating said neurological disorder.
